SEQUENCE LISTING (5 sequences)

SEQ ID NO: 1 - DNA, 70 bp, Artificial Sequence
  A gene included in the DNA library, each of n being a, c, g, or t, where atgc=25%25%25%25%
  misc_feature (21)..(50): n is a, c, g, or t
agcagcacagaggtcagatgnnnnnnnnnnnnnnnnnnnnnnnnnnnnnncctatgcgtgctaccgtgaa

SEQ ID NO: 2 - DNA, 20 bp, Artificial Sequence
  Synthesized primer
ttcacggtagcacgcatagg

SEQ ID NO: 3 - DNA, 20 bp, Artificial Sequence
  Synthesized primer
agcagcacagaggtcagatg

SEQ ID NO: 4 - DNA, 32 bp, Artificial Sequence
  Synthesized aptamer capable of binding to cocaine
agacaaggaaaatccttcaatgaagtgggtcg

SEQ ID NO: 5 - DNA, 21 bp, Artificial Sequence
  Single-stranded DNA capable of forming guanine quadruplex
gggttagggttagggttaggg

(19) United States
(12) Patent Application Publication
(10) Pub. No.: US 2018/0149716 A1
(43) Pub. Date: May 31, 2018

(54) METHOD FOR ACQUIRING APTAMER CAPABLE OF SPECIFICALLY BINDING TO LOW MOLECULAR NITROGEN-CONTAINING ORGANIC COMPOUND BY ELECTROPHORESIS METHOD

(71) Applicant: Panasonic Intellectual Property Management Co., Ltd., Osaka (JP)

(72) Inventor: MARI ONODERA, Osaka (JP)

(21) Appl. No.: 15/602,150

(22) Filed: May 23, 2017

(30) Foreign Application Priority Data
Nov. 28, 2016 (JP) 2016-229731
Jan. 5, 2017 (JP) 2017-000326

(51) Int. Cl. G01R 33/12; G01N 27/327; C12Q 1/68; G01N 27/74

(52) U.S. Cl. CPC G01R 33/1269; G01N 27/3275; C12Q 2600/00; G01N 27/745; C12Q 1/68; C12Q 1/686

(57) ABSTRACT

The present invention provides a method for acquiring an aptamer capable of specifically binding to the low molecular nitrogen-containing organic compound. The method comprises (a) preparing an aqueous solution containing the low molecular nitrogen-containing organic compound and a DNA library; (b) fractionating the aqueous solution by electrophoresis using an electroosmotic-flow-eliminated capillary to provide a fraction containing a composite including the low molecular nitrogen-containing organic compound and a single-stranded DNA contained in the DNA library; and (c) amplifying the single-stranded DNA included in the composite by a PCR method to acquire the amplified gene as the aptamer capable of specifically binding to the low molecular nitrogen-containing organic compound. The present invention provides a method for acquiring an aptamer capable of specifically binding to the low molecular nitrogen-containing organic compound contained in a DNA library by an electrophoresis method.

### INCORPORATION BY REFERENCE-SEQUENCE LISTING

The material contained in the ASCII text file named “P1004889US01_ST25.txt” created on Mar. 21, 2017 and having a file size of 1,695 bytes is incorporated by reference herein.

### BACKGROUND

### 1. Technical Field

The present invention relates to a method for acquiring an aptamer capable of specifically binding to a low molecular nitrogen-containing organic compound by an electrophoresis method.

### 2. Description of the Related Art

An aptamer means a nucleic acid molecule capable of specifically binding to a predetermined molecule. Non-patent Literature 1 discloses an aptamer represented by 5′-HS-(CH₂)₁₁-AGACAAGGAAAATCCTTCAATGAAGTGGGTCG-(CH₂)₇-MB-3′ (SEQ ID NO: 04, “MB” represents methylene blue). The aptamer disclosed in Non-patent Literature is capable of specifically binding to cocaine.

Patent Literature 1 discloses a method for detecting a single nucleotide mutant.

### CITATION LIST

Patent Literature 1

Japanese Patent unexamined Publication No. 2009-38983A

Non-Patent Literature 1

James S. Swensen et al, “Continuous, Real-Time Monitoring of cocaine in Undiluted Blood Serum via a Microfluidic, Electrochemical Aptamer-Based Sensor”, Journal of American Chemical Society, 2009, 131, 4262-4266

Non-Patent Literature 2

Hidenobu Yaku et., al., “Anionic phthalocyanines targeting G-quadruplexes and inhibiting telomerase activity in the presence of excessive DNA duplexes”, Chem. Commun., 2010, Vol. 46, pp. 5740-5742

Non-Patent Literature 3

Richard T. Wheelhouse et. al. “Cationic Porphyrins as Telomerase Inhibitors: the Interaction of Tetra-(N-methyl-4-pyridyl)porphine with Quadruplex DNA”, Journal of the American Chemical Society, 1998, 120, 3261-3262

### SUMMARY

An object of the present invention is to provide a method for acquiring an aptamer capable of specifically binding to a low molecular nitrogen-containing organic compound from a DNA library by an electrophoresis method.

The present invention provides a method for acquiring an aptamer capable of specifically binding to a low molecular nitrogen-containing organic compound, the method comprising:
(a) preparing an aqueous solution containing the low molecular nitrogen-containing organic compound and a DNA library;
(b) fractionating the aqueous solution by electrophoresis using an electroosmotic-flow-eliminated capillary to provide a fraction containing a composite including the low molecular nitrogen-containing organic compound and a single-stranded DNA contained in the DNA library; and
(c) amplifying the single-stranded DNA included in the composite by a PCR method to acquire the amplified single-stranded DNA as the aptamer capable of specifically binding to the low molecular nitrogen-containing organic compound.

The present invention provides a method for acquiring an aptamer capable of specifically binding to a low molecular nitrogen-containing organic compound from a DNA library by an electrophoresis method.

### BRIEF DESCRIPTION OF THE DRAWINGS

FIG. 1 shows a schematic view of a capillary electrophoresis device 100 according to the embodiment.

FIG. 2 shows a schematic view of the step (a) and the step (b) included in the present invention.

FIG. 3 shows a schematic view of the step (c) included in the present invention.

FIG. 4 shows a schematic view of the step (d) included in the present invention.

FIG. 5 shows an electropherogram in the first round of the inventive example 1.

FIG. 6 shows an electropherogram in the sixth round of the inventive example 1.

FIG. 7 shows an electropherogram in the eighth round of the inventive example 1.

FIG. 8 shows an electropherogram in the eleventh round of the inventive example 1.

FIG. 9 shows an electropherogram in the twelfth round of the inventive example 1.

FIG. 10 shows an electropherogram in the verification procedure 1.

FIG. 11 shows an electropherogram in the verification procedure 2.

FIG. 12 shows an electropherogram in the verification procedure 3.

FIG. 13 shows an electropherogram in the verification procedure 4.

FIG. 14 shows an electropherogram in the comparative example 1.

FIG. 15 shows an electropherogram in the reference example 1 in a case where the observation absorption wavelength is 260 nanometers.

FIG. 16 shows an electropherogram in the reference example 1 in a case where the observation absorption wavelength is 424 nanometers.

FIG. 17 shows an electropherogram in the comparative example 2 in a case where the observation absorption wavelength is 260 nanometers.

FIG. 18 shows an electropherogram in the comparative example 2 in a case where the observation absorption wavelength is 424 nanometers.

FIG. 19 shows an electropherogram in the reference comparative example 1.

FIG. 20 shows an electropherogram in the comparative example 3.

FIG. 21 shows an electropherogram in the comparative example 4.

FIG. 22A shows a schematic view of a behavior of a composite 601 in a general electrophoresis capillary.

FIG. 22B shows a schematic view of the behavior of the composite 601 in an electroosmotic-flow-eliminated capillary.

FIG. 23 shows a schematic view of the composite in a case where an organic compound is a high molecular compound 502y.

FIG. 24A shows a schematic view of the behavior of the composite 601 in a case where a nitrogen atom is not included in the organic compound.

FIG. 24B shows a schematic view of a behavior of a single-stranded DNA 504 which is not capable of binding to the organic compound in an electroosmotic-flow-eliminated capillary.

### DETAILED DESCRIPTION OF THE EMBODIMENT

Hereinafter, the present invention will be described in detail with reference to the drawings.

(Step (a))

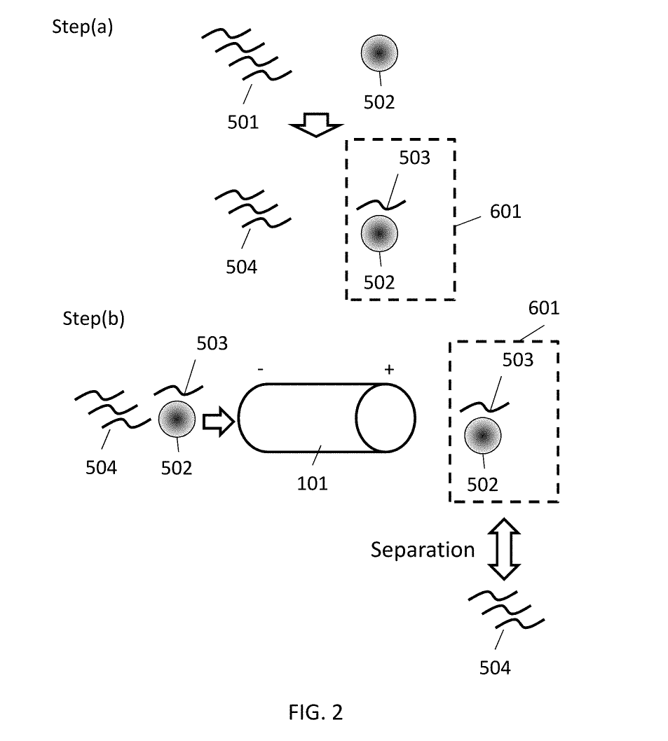

As shown in FIG. 2, first, a low molecular nitrogen-containing organic compound is mixed with a DNA library 501. In FIG. 2, the referential sign 502 represents the low molecular nitrogen-containing organic compound. In this way, an aqueous solution of the low molecular nitrogen-containing organic compound 502 and the DNA library 501 is prepared. The DNA library 501 contains an extremely large number of single-stranded DNAs having different sequences. As one example, the DNA library 501 contains 4³⁰ kinds (approximately 1.15×10¹⁸ kinds) of single-stranded DNAs. A small portion of the extremely large number of the single-stranded DNAs may function as an aptamer(s) capable of specifically binding to the low molecular nitrogen-containing organic compound 502. Hereinafter, the referential sign 503 represents an aptamer capable of specifically binding to the low molecular nitrogen-containing organic compound 502. In addition, in the present specification, the term “aptamer 503” means an aptamer capable of specifically binding to the low molecular nitrogen-containing organic compound 502.

Such a single-stranded DNA which may function as the aptamer 503 bind to the low molecular nitrogen-containing organic compound 502 in the step (a). In this way, a composite 601 including the low molecular nitrogen-containing organic compound 502 and the aptamer 503 is formed. Single-stranded DNAs 504 each of which does not function as an aptamer 503 do not bind to the low molecular nitrogen-containing organic compound 502. As is clear from this description, the aqueous solution contains the low molecular nitrogen-containing organic compound 502, the aptamer 503, and the single-stranded DNAs 504.

(Step (b))

The step (b) is performed after the step (a). In the step (b), the aqueous solution prepared in the step (a) is fractionated through electrophoresis with an electroosmotic-flow-eliminated capillary.

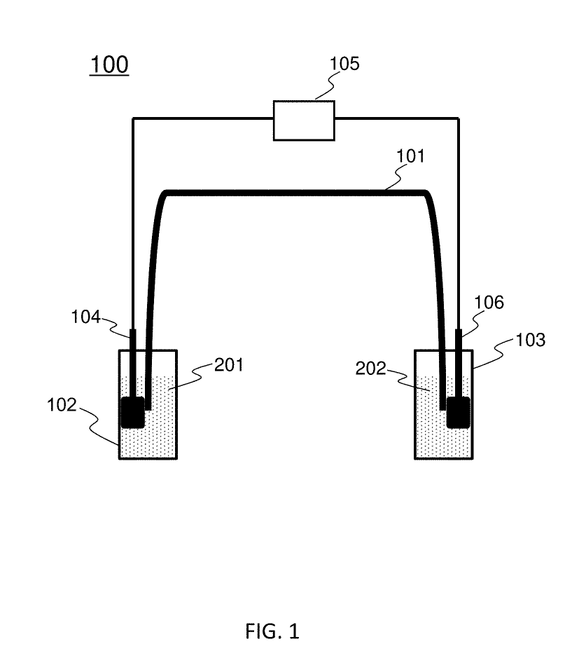

FIG. 1 shows a schematic view of a capillary electrophoresis device 100 according to the embodiment. The capillary electrophoresis device 100 comprises a power supply 105, an electroosmotic-flow-eliminated capillary 101, a first electrode 104, a second electrode 106, a first container 102, and a second container 103.

The electroosmotic-flow-eliminated capillary 101 is a capillary for electrophoresis. A polymer has bound semipermanently to the surface of the inner wall of the electroosmotic-flow-eliminated capillary 101. The polymer eliminates an activity of a silanol group of the inner wall of the capillary to prevent a sample from being adsorbed on the inner wall. In this way, electroosmotic flow is eliminated in the electroosmotic-flow-eliminated capillary. In other words, the electroosmotic-flow-eliminated capillary 101 is a polymer-coated electrophoresis capillary, namely, an electrophoresis capillary having an inner wall coated with a polymer. The electroosmotic-flow-eliminated capillary 101 is commercially available from Agilent Technologies Ltd., trade name: CEP coated capillary.

The inside of the electroosmotic-flow-eliminated capillary 101 is filled with an electrophoretic medium. An example of the electrophoretic medium is a buffer solution such as Tris-HCl.

The present invention is characterized by electrophoresis with the electroosmotic-flow-eliminated capillary 101. As is clear from the inventive examples and the comparative examples which will be described later, in case where the electroosmotic-flow-eliminated capillary 101 is not used, the aptamer capable of specifically binding to the low molecular nitrogen-containing organic compound fails to be obtained.

(First Container 102 and First Electrode 104)

The first container 102 contains a first electrolyte 201. One end of the electroosmotic-flow-eliminated capillary 101 is immersed in the first electrolyte 201. The first container 102 comprises the first electrode 104. The first electrode 104 is in contact with the first electrolyte 201. The first electrode 104 is electrically connected to the power supply 105. An example of the material of the first electrode 104 is titanium, chrome, tungsten, copper, aluminum, platinum, or an alloy thereof. It is desirable that the first electrode 104 has a shape of a flat plate. The material of the first container 102 is not limited, as long as the first container does not react with an electrophoretic medium and the first electrolytes 201. An example of the material of the first container 102 is glass or resin (e.g., polystyrene or polymethylmethacrylate).

(Second Container 103 and Second Electrode 106)

Similarly, the second container 103 contains a second electrolyte 202. The other end of the electroosmotic-flow-eliminated capillary 101 is immersed in the second electrolyte 202. The second container 103 comprises the second electrode 106. The second electrode 106 is in contact with the second electrolyte 202. The second electrode 106 is electrically connected to the power supply 105. Examples of the material and the shape of the second electrode 106 are similar to those of the first electrode 104. An example of the material of the second container 103 is also similar to that of the first container 102.

(Electroosmotic-Flow-Eliminated Capillary 101)

The electroosmotic-flow-eliminated capillary 101 is filled with the electrophoretic medium. It is desirable that the material of the electrophoretic medium is the same as the materials of the first electrolyte 201 and the second electrolyte 202. As one example, in a case where the electroosmotic-flow-eliminated capillary 101 is filled with a sodium carbonate aqueous solution having a concentration of 10 mM, each of the first electrolyte 201 and the second electrolyte 202 is also a sodium carbonate aqueous solution having a concentration of 10 mM.

An example of the electrophoretic medium with which the electroosmotic-flow-eliminated capillary 101 is filled is a buffer solution, an aqueous solution, or pure water. An example of the buffer solution is a boric acid buffer, a phosphate buffer, or a Tris-HCl buffer. The aqueous solution may contain potassium ions, sodium ions, magnesium ions, chloride ions, or carbonate ions. Another example of the aqueous solution is an aqueous solution containing glycine.

The electroosmotic-flow-eliminated capillary 101 may be filled with the mixture of two or more kinds of electrophoretic mediums. In this case, each of the first electrolyte 201 and the second electrolyte 202 is also composed of the same mixture.

(Power Supply 105)

The power supply 105 is located electrically between the first electrode 104 and the second electrode 106. Using the power supply 105, a direct-current voltage is applied between the first electrode 104 and the second electrode 106. The direct-current voltage may have a voltage of not less than 2,000 volts and not more than 30,000 volts.

As well known, genes have negative electric charge. The negative electric charge of the single-stranded DNA bound to the low molecular nitrogen-containing organic compound (namely, the aptamer capable of specifically binding to the low molecular nitrogen-containing organic compound) is decreased to some extent due to the binding to the low molecular nitrogen-containing organic compound. In other words, the composite 601 has smaller negative electric charge than the single-stranded DNAs 504 which have not been bound to the low molecular nitrogen-containing organic compound 502.

As shown in FIG. 1, the aqueous solution is supplied from a cathode side. In the electroosmotic-flow-eliminated capillary 101, the single-stranded DNAs 504 go forward toward an anode side sooner than the composite 601 due to a repulsive force against the cathode and an attracting force toward the anode. This is because the single-stranded DNAs 504 has the larger negative electric charge than the composite 601. In this way, a fraction containing the composite 601 is obtained. In other words, the composite 601 is separated from the single-stranded DNAs 504. As is clear from the comparative example which will be described later, in case where the electroosmotic-flow-eliminated capillary 101 is not used, the composite 601 fails to be separated from the single-stranded DNAs 504. This matter will be described later.

(Step (c))

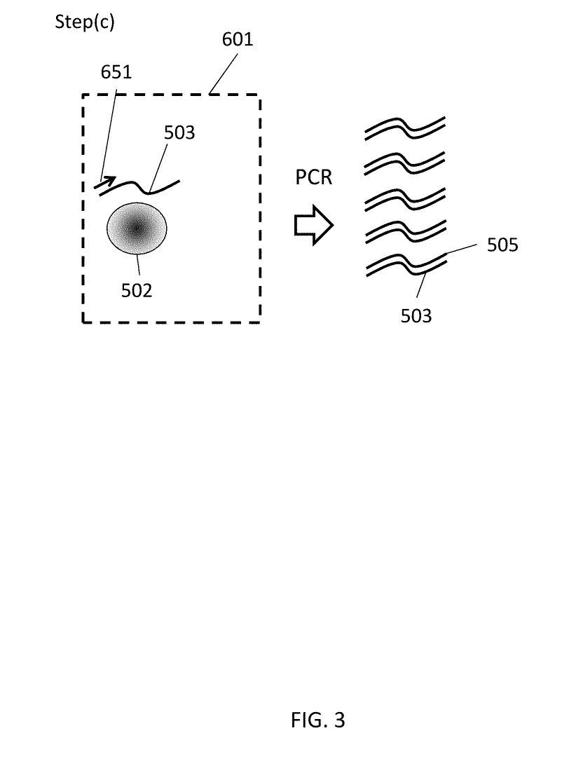

The step (c) is performed after the step (b). As shown in FIG. 3, in the step (c), the single-stranded DNA (i.e., the aptamer 503) included in the composite 601 is amplified by a PCR method. The referential sign 651 indicates a primer used in the PCR method. The thus-amplified single-stranded DNAs are acquired as the aptamer 503. Needless to say, a complementary strand 505 composed of the complementary sequence to the single-stranded DNA (i.e., the aptamer 503) is also amplified by the PCR method. As described later, it is desirable that a biotin-modified primer is used in the PCR method.

(Step (d))

It is desirable that the step (d) is performed after the step (c). In the step (d), the aptamer 503 acquired in the step (c) is purified. Upon the completion of the step (c), an aqueous solution containing not only the aptamer 503 but also other various materials is provided. An example of other various materials (i.e., impurities) contained in the aqueous solution is the low molecular nitrogen-containing organic compound 502, a part of the single-stranded DNAs 504 which have not been separated (namely, removed) sufficiently in the step (b), or unreacted materials in the PCR method. An example of the unreacted materials in the PCR method is an unreacted primer (including the unreacted biotin-modified primer), an unreacted dNTP, or an unreacted enzyme.

The present invention includes the following two purification method.
(i) a method by which a magnetic bead is used.
(ii) a method by which the step (a)-the step (c) are substantially repeated using the aqueous solution provided upon the completion of the step (c) as the DNA library.

Hereinafter, first, the method (i) by which a magnetic bead is used will be described.

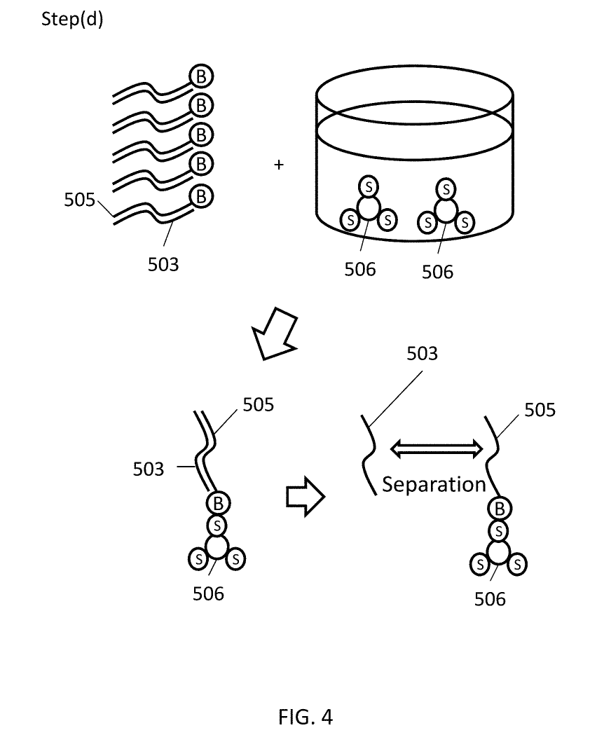

In the method (i), in the PCR method conducted in the step (c), a primer 651 consisting of the gene complementary to a part of the aptamer 503 is modified with one selected from the group consisting of biotin and streptavidin. In FIG. 4, the primer 651 is modified with biotin. It is desirable that an end of the primer 651 is modified. Therefore, two genes are amplified by the PCR method using the primer 651. The amplified one gene is the aptamer 503, and the other amplified gene is the complementary strand 505 consisting of the sequence complementary to the aptamer 503. The complementary strand 505 is modified with the one selected from the group consisting of biotin and streptavidin. Needless to say, each of the aptamer 503 and the complementary strand 505 is a single-stranded DNA. A double-stranded DNA is composed of the aptamer 503 and the complementary strand 505. In other words, the aptamer 503 and the complementary strand 505 are hybridized to each other to form the double-stranded DNA.

(Step (da1))

Then, as shown in FIG. 4, the aptamer 503 and the complementary strand 505 are mixed with a magnetic bead 506. It is desirable that a plurality of the magnetic beads 506 are used. In particular, the aqueous solution containing the double-stranded DNA composed of the aptamer 503 and the complementary strand 505 is mixed with an aqueous solution containing the magnetic bead 506. The magnetic bead 506 has the other selected from the group consisting of biotin and the streptavidin on the surface thereof. In FIG. 4, the magnetic bead 506 has streptavidin on the surface thereof. As well known, biotin and streptavidin are strongly bound to each other. Therefore, the complementary strand 505 modified with biotin is bound to the magnetic bead 506 through the binding of biotin and streptavidin. Since the aptamer 503 is complementary to the complementary strand 505, the aptamer 503 is also bound to the magnetic bead 506 through the complementary strand 505.

(Step (da2) and Step (da3))

Then, the magnetic bead 506 is drawn with magnetism. In particular, a magnet is used. The magnetic bead 506 is drawn to the magnet. Needless to say, at this stage, as shown in FIG. 4, the double-stranded DNA where the aptamer 503 and the complementary strand 505 are hybridized to each other is bound to the surface of the magnetic bead 506. Upon the completion of the step (da1), the aptamer 503 may be bound to the magnetic bead through the complementary strand 505. Alternatively, first, the complementary strand 505 is bound to the surface of the magnetic bead 506 through the binding of biotin and the streptavidin, and then, the aptamer 503 may hybridize to the complementary strand 505. Anyway, the double-stranded DNA composed of the complementary strand 505 and the aptamer 503 is bound to the surface of the magnetic bead 506 drawn with magnetism. Finally, the magnetic bead 506 is collected. In this way, the magnetic bead 506 is separated from impurities.

(Step (da4))

Finally, the aptamer 503 is separated from the double-stranded DNA bound to the collected magnetic bead 506. In particular, an alkaline aqueous solution is added to the double-stranded DNA. In this way, the aptamer 503 is purified.

Then, the method (ii) will be described. In the method (ii), the steps (a)-(c) are substantially repeated using the aqueous solution provided upon the completion of the step (c) as the DNA library.

In particular, the aptamer 503 acquired in the step (c) is mixed with the low molecular nitrogen-containing organic compound 502 similarly to the case of the step (a) to provide an aqueous solution. Then, similarly to the case of the step (b), the aqueous solution is fractionated through electrophoresis with the electroosmotic-flow-eliminated capillary 101 to provide a fraction containing the composite 601 including the aptamer 503 and the low molecular nitrogen-containing organic compound 502. Finally, similarly to the case of the step (c), the aptamer 503 included in the composite 601 contained in the fraction is amplified by a PCR method. The step (d) may also be repeated.

After the method (i), the method (ii) may be performed. Furthermore, after the method (ii), the method (i) may be performed again. In other words, the method (i) and the method (ii) may be repeated alternately. In this way, the aptamer 503 may be further purified.

In this way, the present invention is also a method for extracting an aptamer capable of specifically binding to the low molecular nitrogen-containing organic compound from a DNA library containing an extremely large number of genes having different sequences.

Hereinafter, the term used in the present specification “low molecular nitrogen-containing organic compound” will be described.

The term “low molecule” means a molecule having a molecular weight of not more than 1,000. Therefore, organic compounds such as proteins, resins, DNAs, or RNAs are not included in the term “low molecular nitrogen-containing organic compound”, even if a nitrogen atom is contained in the inside thereof, since proteins, resins, DNAs, and RNAs are generally polymers having molecular weights more than 1,000.

The term “nitrogen-containing” means, literally, to contain a nitrogen atom. Therefore, a compound represented by the chemical formula CₓH_{y}O_{z} (where x, y, and z are each independently a natural number) is not included in the term “low molecular nitrogen-containing organic compound”, even if it is a low molecular organic compound, since there is no doubt that it does not contain a nitrogen atom.

The term “organic compound” means, literally, a compound including carbon. Therefore, an inorganic compound such as gallium nitride is not included in the term “low molecular nitrogen-containing organic compound”, even if it is a low molecular compound and contains a nitrogen atom. Needless to say, conventionally, inorganic compounds include carbon dioxide represented by the chemical formula CO₂, carbon monoxide represented by the chemical formula CO, hydrogen cyanide represented by the chemical formula HCN, carbon disulfide represented by the chemical formula CS₂, carbon tetrachloride represented by the chemical formula CCl₄, and carbonate represented by the chemical formula Mₓ(CO₃)_{y} (where M is a metal atom).

An example of the low molecular nitrogen-containing organic compound is cocaine, phthalocyanine, porphyrin or a derivative thereof.

Without intending to be limited by theory, the present inventors believe the theory of the present invention as below.

The nitrogen atom contained in the low molecular nitrogen-containing organic compound 502 is present as N⁺ in an aqueous solution due to coordination bond with a proton contained in the aqueous solution. Alternatively, when the low molecular nitrogen-containing organic compound 502 is quaternary ammonium salt or a derivative thereof, the nitrogen atom contained in the low molecular nitrogen-containing organic compound 502 is present as N⁺ in an aqueous solution. Anyway, the low molecular nitrogen-containing organic compound 502 is present in an aqueous solution as a low molecular nitrogen-containing organic cation 502c.

The aptamer 503 specifically binds to the low molecular nitrogen-containing organic cation 502c. In this way, the composite 601 is formed. As well known, a DNA has negative charge 503c due to many phosphate groups included in the inside thereof. Therefore, the aptamer 503 which is one kind of DNAs is also charged negatively.

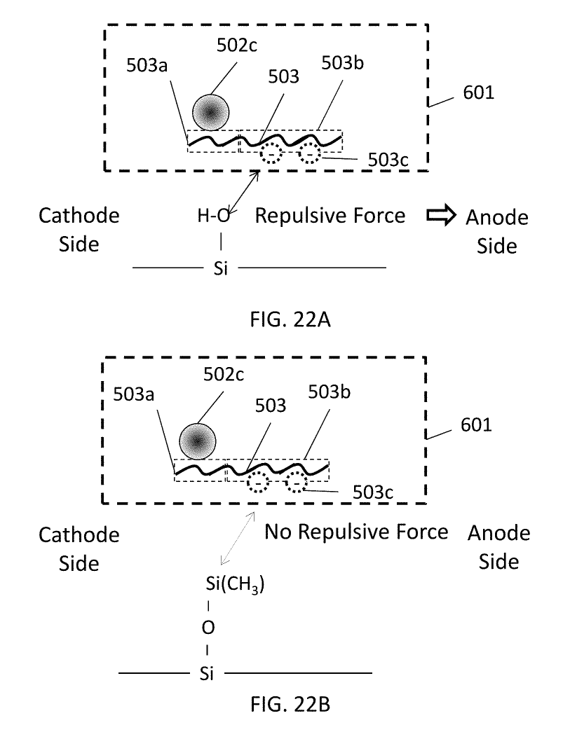

As shown in FIG. 22A and FIG. 22B, in light of molecular weights, the aptamer 503 is much bigger than the low molecular nitrogen-containing organic cation 502c. Therefore, in a part 503a of the aptamer 503 which has been bound to the low molecular nitrogen-containing organic cation 502c, positive charge and negative charge negate each other. However, other part 503b of the aptamer 503 which has not been bound to the low molecular nitrogen-containing organic cation 502c remains to be charged negatively.

The capillary generally used for electrophoresis has a large number of silanol groups represented by the chemical formula Si—OH in the inside thereof. Since the OH group of the silanol group has negative charge, repulsive force is generated between the DNA such as the aptamer and the silanol groups. As shown in FIG. 22A, the repulsive force facilitates the flow of the DNA including the aptamer toward the anode.

On the other hand, in the present invention, the electroosmotic-flow-eliminated capillary is used. As shown in FIG. 22B, in the electroosmotic-flow-eliminated capillary, the silanol group represented by the chemical formula Si—OH is protected with the protecting group such as trimethyl silane or a polymer. Due to the protecting group, the above-mentioned repulsive force does not occur. In other words, in the present invention, such a repulsive force does not occur.

The present inventors believe that this repulsive force has a significant effect on the electrophoresis. The composite 601 moves toward the anode more slowly than the single-stranded DNAs 504 (namely, the single-stranded DNAs 504 which have not been bound to the low molecular nitrogen-containing organic compound 502). This is because the attracting force applied to the composite 601 toward the anode is smaller than attracting force applied to the single-stranded DNAs 504 toward the anode, since a part of the negative charge of the aptamer 503 included in the composite 601 is negated by the low molecular nitrogen-containing organic cation 502c. In other words, between the composite 601 and the single-stranded DNAs 504, there is a difference of the attracting force toward the anode. However, the repulsive force compensates the difference of the attracting force sufficiently. For this reason, the difference of the electrophoresis speed of the composite 601 from that of the single-stranded DNAs 504 included in the capillary used generally for electrophoresis is small. Therefore, in case of using the capillary used generally for electrophoresis, the composite 601 fails to be separated from the single-stranded DNAs 504.

On the other hand, in the present invention, the repulsion does not occur, since the electroosmotic-flow-eliminated capillary is used. Therefore, the difference of the attracting force is not compensated. Therefore, the difference of the electrophoresis speed of the composite 601 from that of the single-stranded DNAs 504 included in the capillary used generally for electrophoresis is big. Therefore, in the present invention, the composite 601 is separated from the single-stranded DNAs 504.

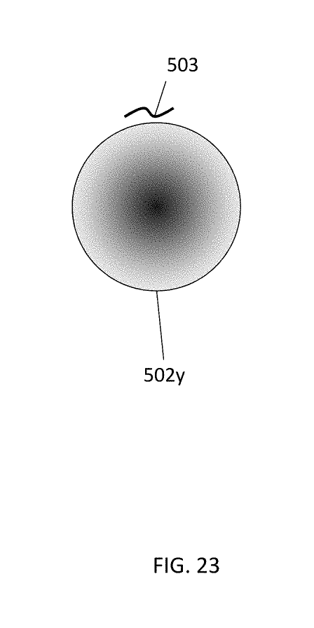

In case of using the high molecular compound 502y in place of the low molecular nitrogen-containing organic compound 502, as shown in FIG. 23, in light of molecular weights, the high molecular compound 502y is much bigger than the aptamer 503. Therefore, the aptamer 503 hardly has an effect on the electrophoresis of the high molecular compound 502y. For this reason, it is meaningless to apply the present invention to the high molecular compound 502y in place of the low molecular nitrogen-containing organic compound 502. In other words, it is easy to separate the high molecular compound 502y of which the aptamer 503 has been adsorbed on the surface from the single-stranded DNAs 504 with the capillary used generally for electrophoresis. See the comparative example 4 which will be described later.

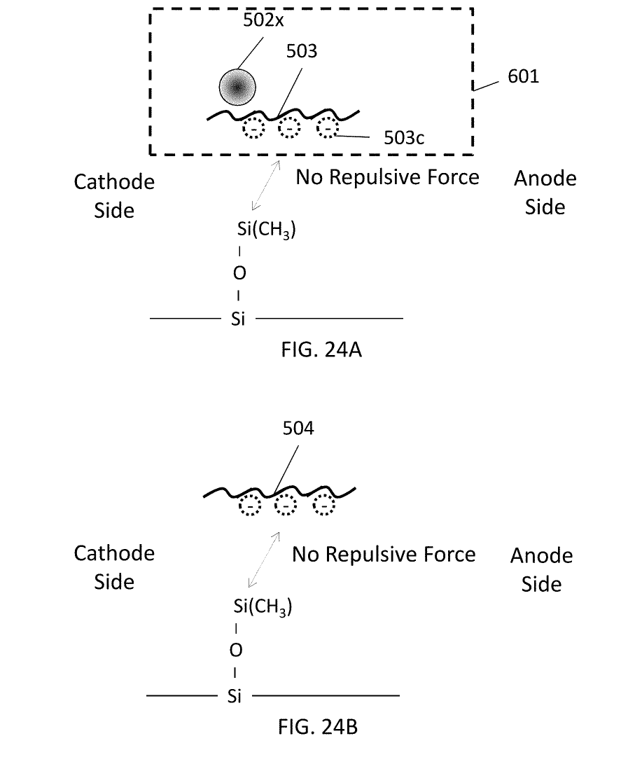

In case where the organic compound does not contain a nitrogen atom, as shown in FIG. 24A and FIG. 24B, since the negative charge of the aptamer 503 included in the composite 601 is not negated by the organic cation 502x, the attracting force and the repulsive force applied to the composite 601 is the same as that of the single-stranded DNAs 504. Therefore, in case where the organic compound does not contain a nitrogen atom, as demonstrated in the comparative example 3 which will be described later, even when the electroosmotic-flow-eliminated capillary is used, the composite 601 fails to be separated from the single-stranded DNAs 504.

The present inventors believe that there is no aptamer capable of binding to a nitrogen-containing inorganic material such as ammonia.

### EXAMPLES

Hereinafter, the present invention will be described in more detail with reference to the following examples.

### Experiment 1

In the experiment 1, cocaine was used as the low molecular nitrogen-containing organic compound 502. Cocaine is a low molecular nitrogen-containing organic compound represented by the chemical formula C₁₇H₂₁NO₄. Cocaine has a molecular weight of approximately 303. The experiment 1 consists of the inventive example 1 (including the verification procedures 1-4) and the comparative example 1. Cocaine was purchased as cocaine hydrochloride.

### Inventive Example 1

The following Table 1 shows details of the electroosmotic-flow-eliminated capillary 101 employed in the inventive example 1. The following Table 2 shows reagents employed in the inventive example 1.

TABLE 1

| Trade name | CEP coated capillary (available from | |
|---|---|---|
| | Agilent Technologies, Ltd.) | |
| Inner diameter | 75 | micrometers |
| Overall length | 48.5 | centimeters |
| effective length | 38 | centimeters |

| | |
|---|---|
| Material | Fused silica |

TABLE 2

| | |
|---|---|
| Electro- | 20 mM Tris-HCl (pH 7.5) |
| phoretic | |
| medium | |
| Electro- | 20 mM Tris-HCl (pH 7.5) |
| phoretic | 10 μM DNA library |
| material | Sequence: 5′- |
| | AGCAGCACAGAGGTCAGATG-(N30)-CCTATGCGT |
| | GCTACCGTGAA-3′ (SEQ ID NO: 01) |
| | N30: 30 base random sequence |
| | (A:T:G:C = 25%:25%:25%25%) |
| | 10 mM Cocaine |
| | The DNA library was purchased from |
| | Tsukuba oligo service Co., Ltd. |
| First | 20 mM Tris-HCl (pH 7.5) |
| electrolyte | |
| Second | 20 mM Tris-HCl (pH 7.5) |
| electrolyte | |

(Electrophoresis)

First, the electrophoretic medium was supplied to the inside of the electroosmotic-flow-eliminated capillary 101 at pressure of 138 kPa for 20 minutes. In this way, the inside of electroosmotic-flow-eliminated capillary 101 was washed. Then, similarly, the inside of electroosmotic-flow-eliminated capillary 101 was filled with the electrophoretic medium.

The DNA library and cocaine were added to a Tris-HCl buffer to prepare the electrophoretic material shown in Table 2 (See FIG. 2). This electrophoretic material was supplied to the inside of the electroosmotic-flow-eliminated capillary 101 filled with the electrophoretic medium at negative pressure of 3.45 kPa for 7.8 seconds. In this way, the inside of electroosmotic-flow-eliminated capillary 101 was filled with the electrophoretic material.

The first electrode 104 was brought into contact with the first electrolyte 201 included in the first container 102. Similarly, the second electrode 106 was brought into contact with the second electrolyte 202 included in the second container 103.

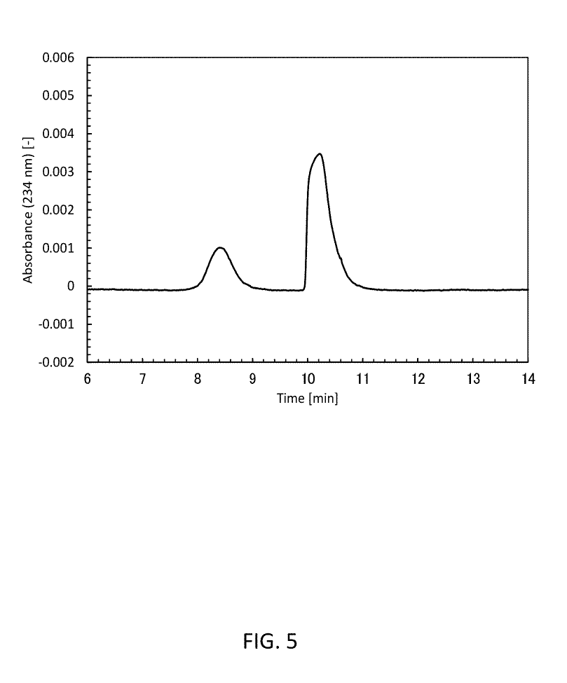

A DC voltage of 12 kV was applied between the first electrode 104 and the second electrode 106 using the power supply 105 (See FIG. 1 and FIG. 2). The first electrode 104 was used as a cathode. The second electrode 106 was used as an anode. While the second container 103 containing the second electrolyte 202 was replaced immediately with another second container 103 every one minute, the electrophoretic material (i.e., the aqueous solution) was fractionated through such an electrophoresis. In this way, a plurality of the fractions were obtained. FIG. 5 shows an electropherogram obtained in this electrophoresis. The observation absorption wavelength in the electropherogram was 234 nanometers. Cocaine has absorption maximum at 234 nanometers.

As shown in FIG. 5, the electropherogram has two peaks. One peak (hereinafter, referred to as “the first peak”) appears between eight minutes and nine minutes. The other peak (hereinafter, referred to as “the second peak”) appears between ten minutes and eleven minutes. Since the negative electric charge of the single-stranded DNA (namely, the aptamer 503 capable of specifically binding to cocaine) bound to cocaine 502 is decreased to some extent due to the binding to the cocaine 502, the present inventor believed that the fraction of the second peak contained the composite 601 including cocaine 502 and the aptamer 503 capable of specifically binding to cocaine.

(PCR)

Then, a mixture (1 milliliter) shown in the following Table 3 was prepared. Note that the mixture contains the fraction of the second peak (100 microliters). The primers represented by the SEQ ID NO: 02 and the SEQ ID NO: 03 were purchased from the Tsukuba oligo service Co., Ltd.

TABLE 3

| | |
|---|---|
| Buffer solution | The buffer solution contained the following materials: |
| | 2 mM Tris-HCl buffer solution (pH 8.0); |
| | 10 mM KCl; |
| | 0.01 mM ethylenediaminetetraacetic acid; |
| | 0.1 mM dithiothreitol; |
| | 0.05% Tween20; |
| | 0.05% poly(oxyethylene) nonyl phenyl ether (purchased from |
| | Nacalai Tesque, Inc. trade name ″Nonidet P-40″); and |
| | 5% glycerol. |
| Biotin-modified | 5′-(biotin)-TTCACGGTAGCACGCATAGG-3′ (1 μM) |
| Reverse | (SEQ ID NO: 02) |
| Primer | |
| Forward | 5′-AGCAGCACAGAGGTCAGATG-3′ (1 μM) |
| Primer | (SEQ ID NO: 03) |
| template DNA | Fraction of the second peak (100 microliter) |
| solution | |
| dNTP | 0.4 mM dNTP (purchased from Takara bio Inc.) |
| Enzyme | Takara LA Taq Hot Start Version (purchased from Takara bio |
| | Inc.) which was diluted 100 times. |

Then, the mixture was subjected to the following PCR method.

First, the mixture was heated at 94 degrees Celsius for 5 minutes.

Subsequently, the temperature of the mixture was changed in accordance with the following cycle.
at 94 degrees Celsius for one minute;
at 55 degrees Celsius for one minute; and
at 72 degrees Celsius for one minute.

This cycle was repeated 40 times.

Finally, the mixture was left at rest at a temperature of 72 degrees Celsius. Then, the mixture was cooled to 4 degrees Celsius and stored.

In this way, a double-stranded DNA was obtained as a PCR product (See FIG. 3). As shown in FIG. 3, the thus-obtained DNA was composed of the aptamer 503 capable of specifically binding to cocaine included in the fraction of the second peak and its complementary strand 505 having 5′-terminal modified with biotin.

(Purification of Aptamer Capable of Specifically Binding to Cocaine)

Next, a dispersion liquid of the magnetic beads 506 each having a surface modified with streptavidin (1 milliliter, purchased from ThermoFisher scientific K. K., trade name: Dynabeads M-280 Streptavidin) was supplied to a microtube. The magnetic beads 506 were washed three times with a buffer solution contained 10 mM Tris-HCl (pH 7.5) and 2M KCl. In particular, a magnet was brought close to the bottom of the microtube. The magnet and the microtube were left at rest for two minutes. Subsequently, a supernatant was removed. This was repeated once again.

Then, the washed magnetic beads 506 were dispersed in a buffer solution (1 milliliter) containing 10 mM Tris-HCl (pH 7.5) and 2M KCl. Furthermore, the mixture (1 milliliter) containing the PCR product was added to the buffer solution. The mixture was stirred at 25 degrees Celsius for 30 minutes at 1,300 rpm. In the mixture, double-stranded DNAs were bound to the surface of the magnetic beads 506 through the binding of biotin-streptavidin shown in FIG. 4.

The magnetic beads 506 were collected again using the magnet. Then, a supernatant was removed. Subsequently, the magnetic beads 506 were washed three times similarly to the above using the buffer solution containing 10 mM Tris-HCl (pH 7.5) and 1M NaCl.

A sodium hydroxide aqueous solution (0.15 M, 1 milliliter) was added to the washed magnetic beads 506 to provide a mixture. The mixture was stirred at 25 degrees Celsius for 30 minutes at 1,300 rpm. In this way, the aptamer 503 capable of specifically binding to cocaine included in the double-stranded DNA was separated from the magnetic beads 506 (See FIG. 4).

Finally, the magnetic beads 506 were collected using the magnet. The magnet and the magnetic beads were left at rest for two minutes. A supernatant containing the aptamer 503 capable of specifically binding to cocaine was collected. In other words, the aptamer 503 capable of specifically binding to cocaine was separated from the magnetic beads 506 and the complementary strand 505. In this way, the aptamer 503 capable of specifically binding to cocaine was purified.

(Drying and Concentration of Aptamer Capable of Specifically Binding to Cocaine)

The supernatant containing the aptamer 503 capable of specifically binding to cocaine was desalinated with a desalination column (purchased from GE Healthcare). The eluate used for the desalination was Milli-Q water (namely, ultra-pure water). The desalinated supernatant was supplied to a microtube. Then, the supernatant was dried under a vacuum. In this way, the Mill-Q water contained in the microtube was evaporated and the aptamer 503 capable of specifically binding to cocaine was concentrated. Finally, Tris-HCl (20 mM, pH 7.5) and cocaine (10 mM) were added to the concentrated aptamer 503 capable of specifically binding to cocaine to provide an aqueous solution (50 microliter) as a product of the first round. The first round includes four processes of the electrophoresis, the PCR, the purification of the aptamer capable of specifically binding to cocaine, and drying and concentration of the aptamer capable of specifically binding to cocaine.

(Further Purification)

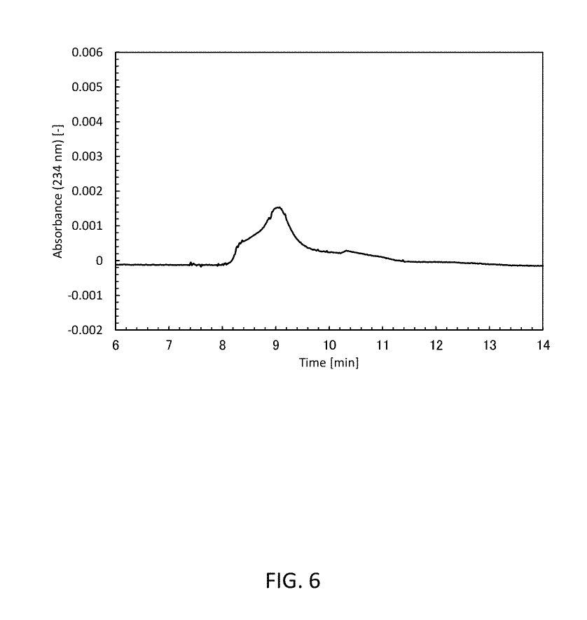

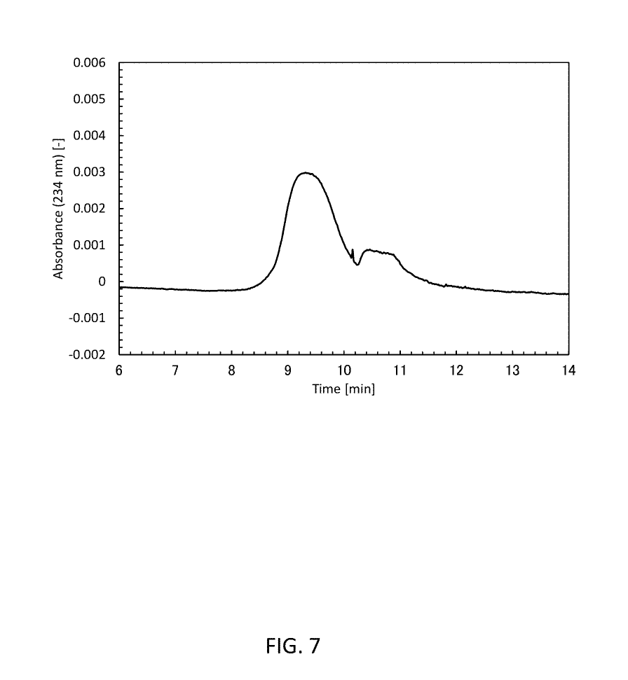

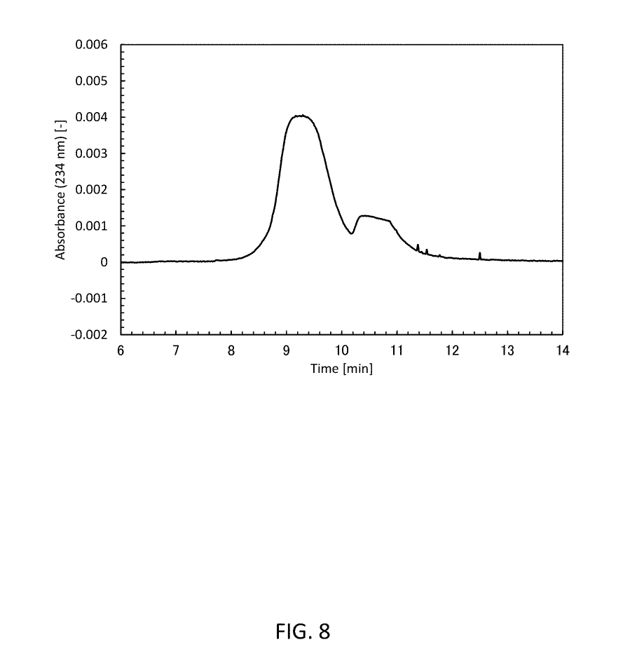

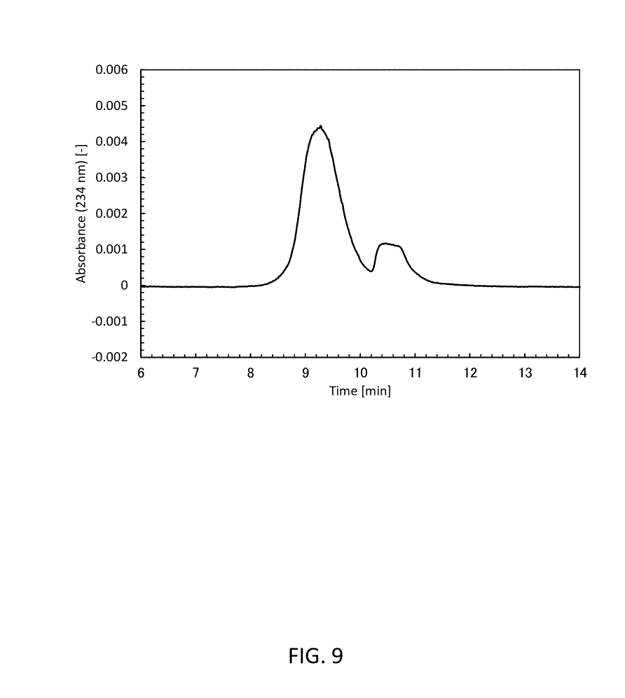

Using the product of the first round as the electrophoretic material, the four processes similar to the first round were repeated as the second round to provide a product of the second round. This was repeated. As a result, the products of the first-twelfth rounds were obtained. In this way, the aptamer 503 capable of specifically binding to cocaine was further purified. FIG. 6 shows an electropherogram in the sixth round. FIG. 7 shows an electropherogram in the eighth round. FIG. 8 shows an electropherogram in the eleventh round. FIG. 9 shows an electropherogram in the twelfth round.

(Verification Procedure 1)

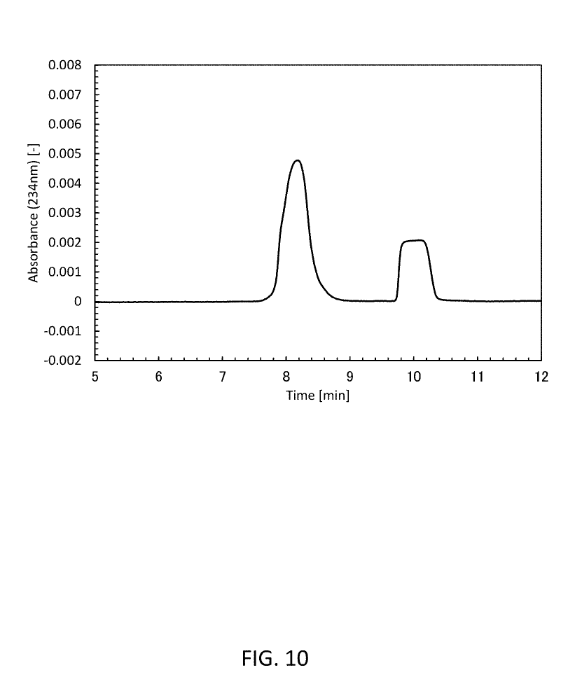

In the verification procedure 1, the product of the twelfth round was mixed with cocaine (concentration: 10 mM) to provide a mixture. The electrophoresis of the mixture was performed again with the electroosmotic-flow-eliminated capillary. FIG. 10 shows an electropherogram in the verification procedure 1.

(Verification Procedure 2)

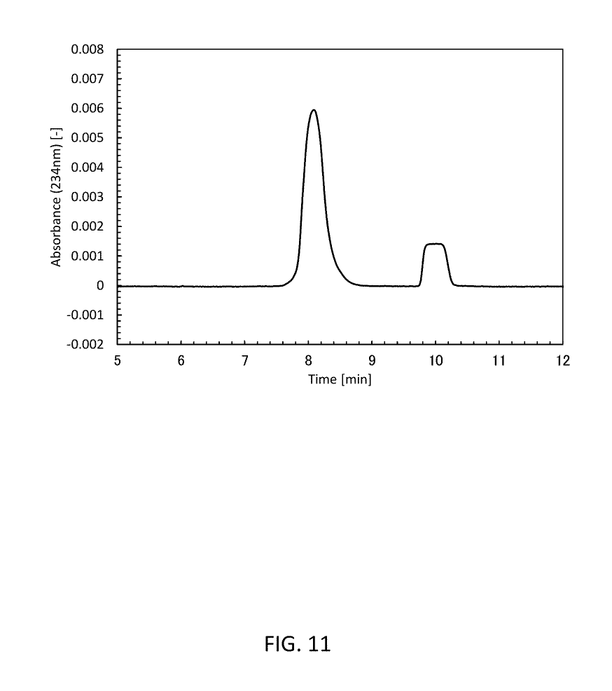

The electrophoresis similar to the verification procedure 1 was performed, except that the concentration of cocaine was 5 mM. FIG. 11 shows an electropherogram in the verification procedure 2.

(Verification Procedure 3)

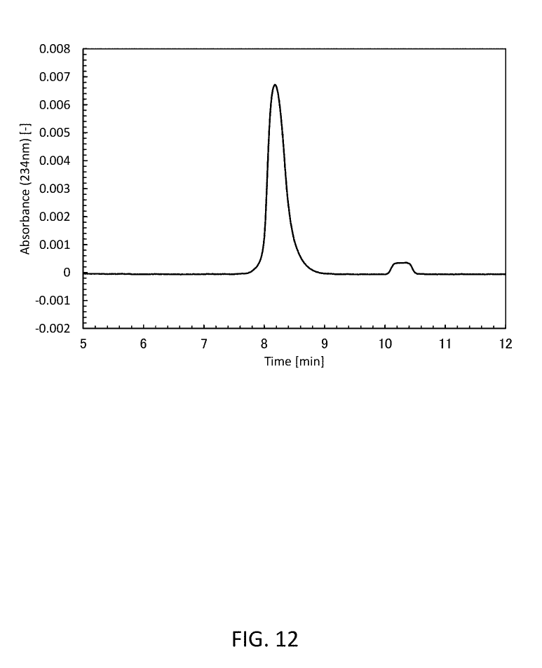

The electrophoresis similar to the verification procedure 1 was performed, except that the concentration of cocaine was 1 mM. FIG. 12 shows an electropherogram in the verification procedure 3.

(Verification Procedure 4)

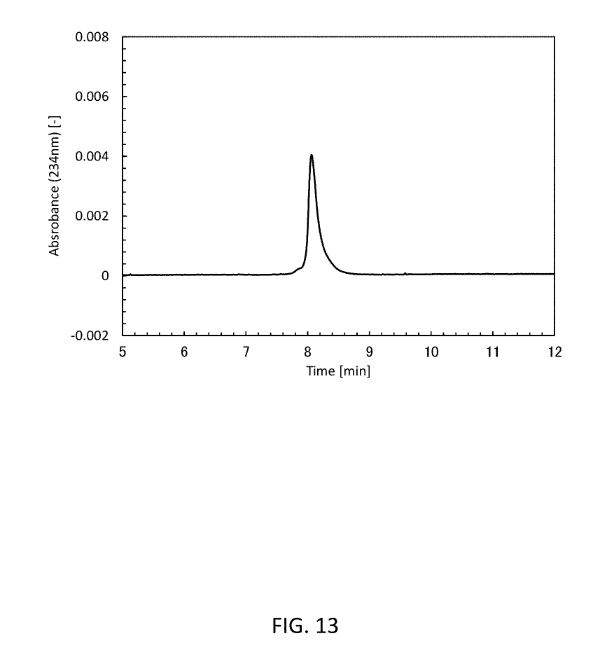

The electrophoresis similar to the verification procedure 1 was performed, except that the concentration of cocaine was 0 mM (namely, no cocaine was added). FIG. 13 shows an electropherogram in the verification procedure 4.

As is clear from the comparison of FIG. 10-FIG. 12 with FIG. 13, a peak was observed near 10 minutes in FIG. 10-FIG. 12, whereas no peak was observed near 10 minutes in FIG. 13. This means that the aptamer contained in the product of the twelfth product was bound to cocaine.

### Comparative Example 1

In the comparative example 1, an experiment similar to the inventive example 1 was performed, except for using a standard fused-silica capillary (purchased from Beckman Coulter, Inc.) in place of the electroosmotic-flow-eliminated capillary 101 (namely, the CEP coated capillary). In this fused-silica capillary, an electroosmotic flow occurs. The following Table 4 shows the details of this fused-silica capillary.

TABLE 4

| Trade name | Fused-silica capillary (purchased from | |
|---|---|---|
| | Beckman Coulter, Inc.) | |
| Inner diameter | 75 | micrometers |
| Overall length | 48.5 | centimeters |
| Effective length | 38 | centimeters |

| | |
|---|---|
| Material | Fused silica |

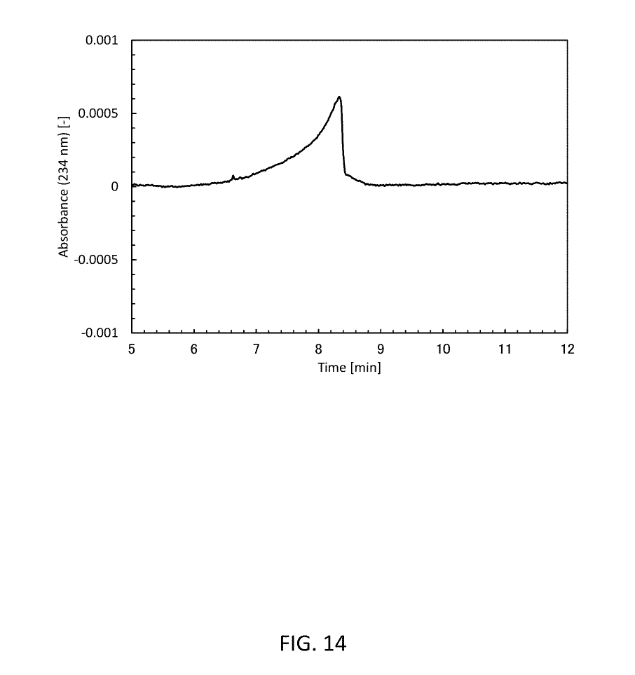

FIG. 14 shows an electropherogram in the comparative example 1. As is clear from FIG. 14, only one peak was observed. This means that the composite 601 failed to be separated from the single-stranded DNAs 504.

### Reference Example 1

In the reference example 1, an experiment similar to the example 1 was conducted, except that the reagents shown in the following Table 5 were used in place of the reagents shown in Table 2. In the reference example 1, copper porphyrin having a molecular weight of approximately 740 was used as the low molecular nitrogen-containing organic compound 502.

TABLE 5

| | |
|---|---|
| Electrophoretic | 100 mM Na₂B₄O₇·10H₂O (pH 9.3) |
| medium | |
| Electrophoretic | 50 mM MES-LiOH (pH 7.0) |
| material | 100 mM KCl |
| | 10 μM Aptamer capable of binding to Cu-TMpyP4 (Molecular |
| | Weight: 740.36) |
| | Sequence: 5′-GGG TTA GGG TTA GGG TTA GGG-3′ |
| | (SEQ ID NO: 05) |
| | 10 μM Cu-TMpyP4 |
| | Cu-TMpyP4 was purchased from Frontier Scientific Inc., |
| | Catalog Number: 41397). |
| | Cu-TMpyP4 means Cu(II) meso-Tetra(N-methyl-4-pyridyl) |
| | porphine tetrachloride (CAS NO: 79407-87-7). |
| | The above aptamer was purchased from Tsukuba oligo |
| | service Co., Ltd. |
| First electrolyte | 100 mM Na₂B₄O₇·10H₂O (pH 9.3) |
| Second | 100 mM Na₂B₄O₇·10H₂O (pH 9.3) |
| electrolyte | |

According to Non-patent Literature 2 and Non-patent Literature 3, a porphyrin skeleton “TMpyP4” is bound to a guanine quadruplex formed of the repeating sequence of “GGG” and “TTA” included in the DNA sequence represented by SEQ ID NO: 05.

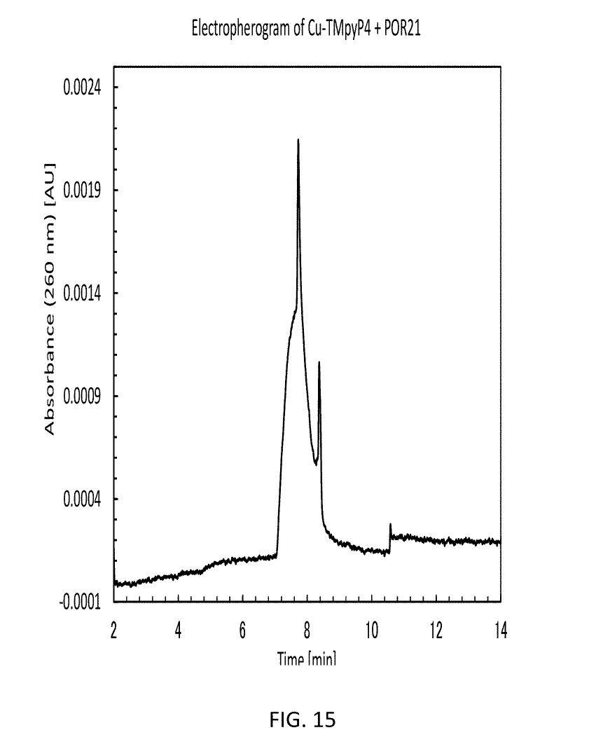

FIG. 15 shows an electropherogram in the reference example 1. FIG. 15 corresponds to FIG. 5. In the reference example 1, the observation absorption wavelength in the electropherogram was 260 nanometers. In other words, FIG. 15 shows an electropherogram in the first round of the reference example 1.

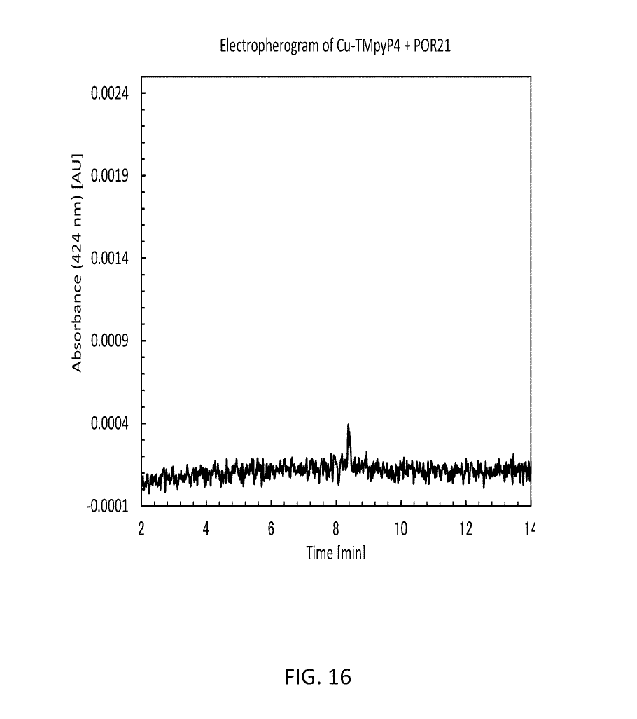

FIG. 16 shows an electropherogram in the reference example 1 in a case where the observation absorption wavelength is 424 nanometers. Cu-TMpyP4 has absorption maximum at 424 nanometers.

### Comparative Example 2

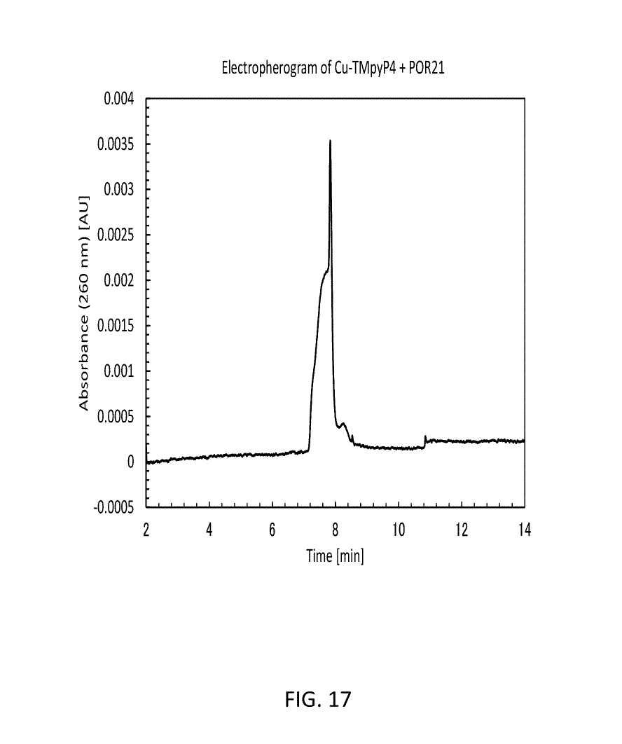

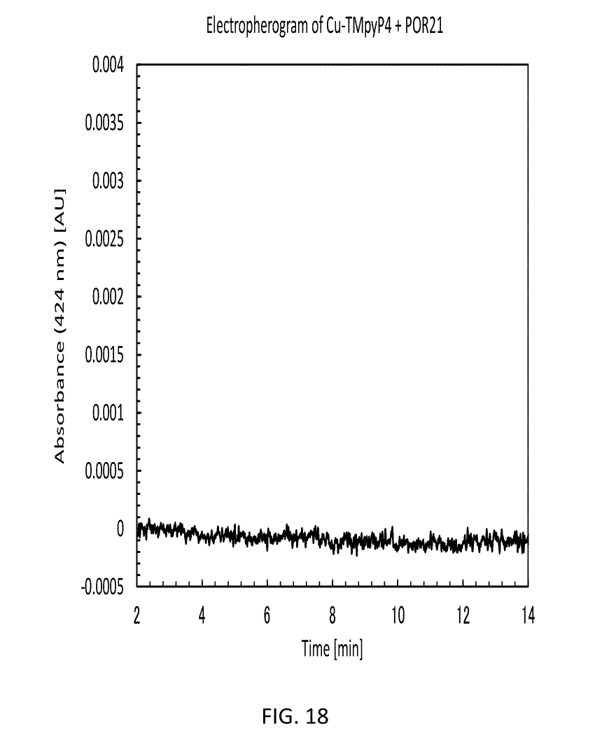

In the comparative example 2, an experiment similar to the reference example 1 was conducted, except that Cu-TMpyP4 was not used. FIG. 17 shows an electropherogram in the comparative example 2 in a case where the observation absorption wavelength is 260 nanometers. FIG. 18 shows an electropherogram in the comparative example 2 in a case where the observation absorption wavelength is 424 nanometers.

As is clear from FIG. 17, in a case where Cu-TMpyP4 was not used, only one peak was observed at the observation absorption wavelength of 260 nanometers. This peak, namely, the first peak located at the time of 7 minutes 40 seconds, corresponds to the DNA aptamer represented by SEQ ID NO: 05.

As is clear from FIG. 18, in a case where Cu-TMpyP4 was not used, no peaks were observed at the observation absorption wavelength of 424 nanometers.

On the other hand, as is clear from FIG. 15, in a case where Cu-TMpyP4 was used, two peaks were observed at the observation absorption wavelength of 260 nanometers.

As is clear from FIG. 16, in a case where Cu-TMpyP4 was used, a peak was observed at the observation absorption wavelength of 424 nanometers. Since the composite of Cu-TMpyP4 and the aptamer has negative charge, the attracting force occurs between the second electrode 106 having positive charge and the composite.

The location of the second peak included in FIG. 15 accords with the location of the peak included in FIG. 16 at the time of 8 minutes 20 seconds. Therefore, the second peak included in FIG. 15 corresponds to the composite of Cu-TMpyP4 and the aptamer.

As described above, the first and second peaks included in FIG. 15 correspond respectively to the DNA aptamer and the composite (namely, the composite of the Cu-TMpyP4 and the aptamer). Therefore, in the reference example 1, the composite of the Cu-TMpyP4 and the aptamer was separated from the single-stranded DNAs 504.

### Reference Comparative Example 1

In the reference comparative example 1, an experiment similar to the reference example 1 was conducted, except for using the standard fused-silica capillary (purchased from Beckman Coulter, Inc., see Table 4 for more detail) in place of the electroosmotic-flow-eliminated capillary 101 (namely, the CEP coated capillary). In this fused-silica capillary, an electroosmotic flow occurs.

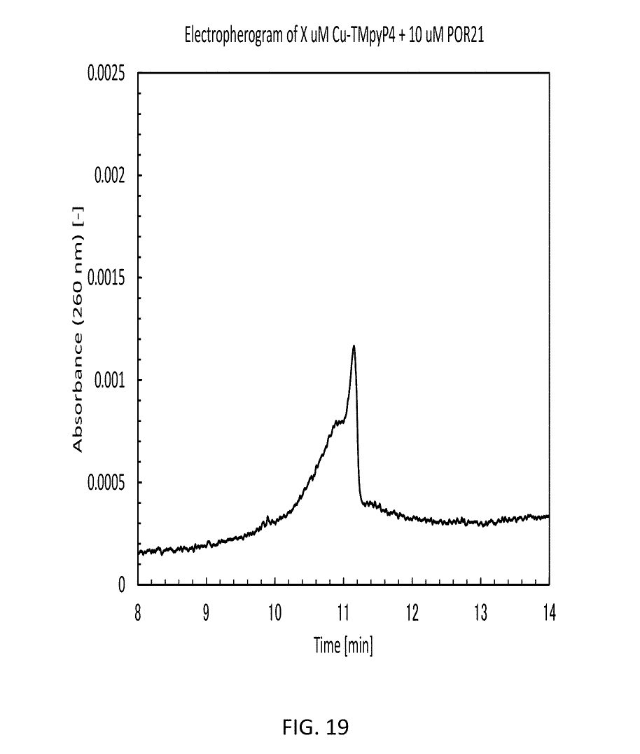

FIG. 19 shows an electropherogram in the reference comparative example 1. As is clear from FIG. 19, only one peak was observed. This means that the composite of Cu-TMpyp4 and the aptamer failed to be separated from the single-stranded DNAs 504.

### Comparative Example 3

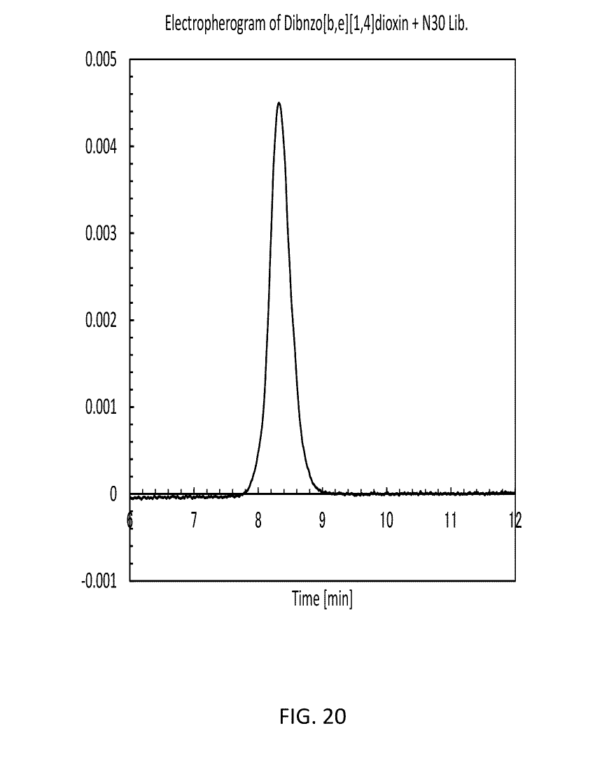

In the comparative example 3, an experiment similar to the inventive example 1 was conducted, except of using dibenzo[b,e][1,4]dioxin represented by the chemical formula C₁₂H₈O₂ (CAS NO: 262-12-4) in place of cocaine. FIG. 20 shows an electropherogram in the comparative example 3. As is clear from FIG. 20, only one peak was observed. This means that the complex of the dibenzo[b,e][1,4]dioxin and the aptamer failed to be separated from the single-stranded DNAs 504.

### Comparative Example 4

In the comparative example 4, an experiment similar to the inventive example 1 was conducted, except for the following matters (I) and (II).

(I) Thrombin from human plasma was used in place of cocaine. (Concentration: 10 μM, Molecular weight; approximately 37.4 kDa, purchased from Sigma Aldrich).

(II) The standard fused-silica capillary (purchased from Beckman Coulter, Inc., see Table 4 for more detail) was used in place of the electroosmotic-flow-eliminated capillary 101 (namely, the CEP coated capillary)

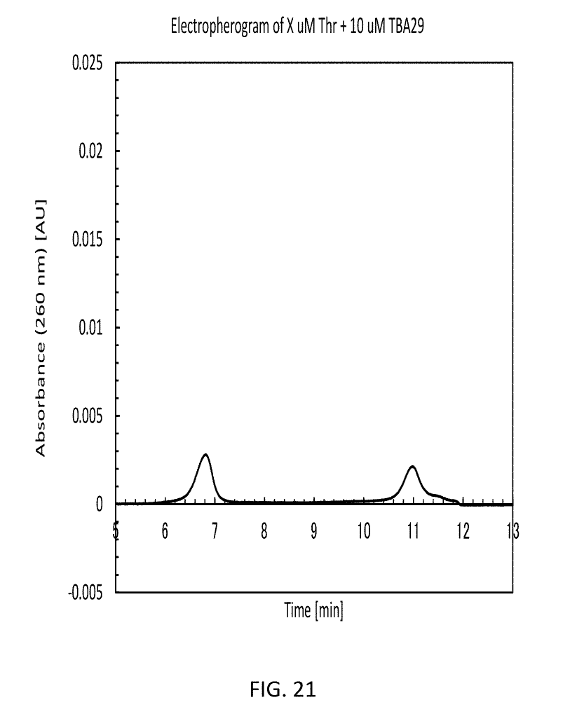

FIG. 21 shows an electropherogram in the comparative example 4. As is clear from FIG. 21, two peaks were observed. This means that a composite of a high molecular compound such as thrombin and an aptamer is easily separated from the single-stranded DNAs 504 with a standard fused-silica capillary (purchased from Beckman Coulter, Inc., see Table 4 for more detail).

### INDUSTRIAL APPLICABILITY

The aptamer provided according to the present invention can be used for the detection of the low molecular nitrogen-containing organic compound.

### REFERENTIAL SIGNS LIST

100 Capillary electrophoresis device
101 Electroosmotic-flow-eliminated capillary
102 First container
103 Second container
104 First electrode
105 Power supply
106 Second electrode
201 First electrolyte
202 Second electrolyte
501 DNA library
502 Low molecular nitrogen-containing organic compound
503 Aptamer capable of specifically binding to the low molecular nitrogen-containing organic compound

### 504 Single-stranded DNAs

505 Complementary strand

### 601 Composite

### 651 Primer

1. A method for acquiring an aptamer capable of specifically binding to a low molecular nitrogen-containing organic compound, the method comprising:
(a) preparing an aqueous solution containing the low molecular nitrogen-containing organic compound and a DNA library;
(b) fractionating the aqueous solution by electrophoresis using an electroosmotic-flow-eliminated capillary to provide a fraction containing a composite including the low molecular nitrogen-containing organic compound and a single-stranded DNA contained in the DNA library; and
(c) amplifying the single-stranded DNA included in the composite by a PCR method to acquire the amplified single-stranded DNA as the aptamer capable of specifically binding to the low molecular nitrogen-containing organic compound.

2. The method according to claim 1, further comprising:
(d) purifying the aptamer capable of specifically binding to the low molecular nitrogen-containing organic compound acquired in the step (c).

3. The method according to claim 2, wherein
in the PCR method in the step (c), a primer modified with one selected from the group consisting of biotin and streptavidin is used;
in the PCR method in the step (c), not only the single-stranded DNA but also a complementary strand composed of a sequence complementary to the single-stranded DNA is amplified;
the amplified complementary strand is modified with the one selected from the group consisting of biotin and streptavidin; and
the step (d) comprises the following substeps:
(da1) mixing a magnetic bead having the other selected from the group consisting of biotin and streptavidin on the surface thereof with the amplified single-stranded DNA and the amplified complementary strand to bind the amplified complementary strand to the surface of the magnetic bead through the binding of biotin and streptavidin;
(da2) drawing the magnetic bead with magnetism; wherein in the substep (da2),
a double-stranded DNA where the amplified complementary strand and the amplified single-stranded DNA are hybridized to each other are bound to the surface of the magnetic bead; and
the amplified complementary strand is bound to the surface of the magnetic bead through the binding of biotin and streptavidin;
(da3) collecting the magnetic bead; and
(da4) separating the amplified single-stranded DNA from the double-stranded DNA bound to the surface of the collected magnetic bead to purify the amplified single-stranded DNA as the aptamer capable of specifically binding to the low molecular nitrogen-containing organic compound.

4. The method according to claim 3, wherein
in the substep (da4), an alkaline aqueous solution is added to the double-stranded DNA to separate the amplified single-stranded DNA.

5. The method according to claim 2, wherein
the step (d) comprises the following substeps:
(db1) mixing the aptamer capable of specifically binding to the low molecular nitrogen-containing organic compound acquired in the step (c) with the low molecular nitrogen-containing organic compound to provide an aqueous solution;
(db2) fractionating the aqueous solution provided in the step (db1) by electrophoresis using an electroosmotic-flow-eliminated capillary to provide a fraction containing a composite including the low molecular nitrogen-containing organic compound and a single-stranded DNA contained in the aqueous solution provided in the step (db1); and
(db3) amplifying the single-stranded DNA included in the composite contained in the fraction provided in the step (db2) by a PCR method to purify the amplified single-stranded DNA as the aptamer capable of specifically binding to the low molecular nitrogen-containing organic compound.

6. A method for extracting, from a DNA library, an aptamer capable of specifically binding to a low molecular nitrogen-containing organic compound contained in the DNA library, the method comprising:
fractionating an aqueous solution containing the DNA library and the low molecular nitrogen-containing organic compound by electrophoresis using an electroosmotic-flow-eliminated capillary to provide a fraction containing a composite composed of the low molecular nitrogen-containing organic compound and the aptamer capable of specifically binding to the low molecular nitrogen-containing organic compound contained in the DNA library.

7. The method according to claim 1, wherein
the low molecular nitrogen-containing organic compound is cocaine.

8. The method according to claim 1, wherein
the low molecular nitrogen-containing organic compound is one selected from the group consisting of phthalocyanine, porphyrin, and a derivative thereof.
